(19) United States
(12) Patent Application Publication
(10) Pub. No.: US 2009/0191531 A1
(43) Pub. Date: Jul. 30, 2009

(54) Method and Apparatus for Integrating Audio and/or Video With a Book

(76) Inventors: Joseph Saccocci, (US); G. Randy Duensing, (US)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/341,761

(22) Filed: Dec. 22, 2008

Related U.S. Application Data
(60) Provisional application No. 61/016,396, filed on Dec. 21, 2007.

(51) Int. Cl. G09B 5/00; A61M 21/02

(52) U.S. Cl. 434/317; 600/27; 600/26; 345/901; 348/61

(57) ABSTRACT

The subject invention relates to a method and apparatus for synchronization of sensory stimulation with the reading experience. In a specific embodiment, audio and/or video stimulation can be provided to a reader. In additional embodiments, the sensory stimulation can include touch, smell, and/or taste. The reading experience can include, for example, the reading of books, magazines, textbooks, telephone directories, maps, resumes, brochures, newspapers, or other forms of written or printed materials, which can include, but are not limited to, words, photographs, illustrations, drawings, cartoons, and braille. The synchronization of sensory stimulation with the reading experience can involve providing sensory stimulation in relation to a reader's location or approximate location with respect to written or printed materials. In addition to sensory stimulation, other forms of interaction can be provided, such as interactive games, or interactive tests. A specific embodiment relates to a method and apparatus for synchronizing audio and/or video to a book as it is read.

### CROSS-REFERENCE TO RELATED APPLICATION

The present application claims the benefit of U.S. Provisional Application Ser. No. 61/016,396, filed Dec. 21, 2007, which is hereby incorporated by reference herein in its entirety, including any figures, tables, or drawings.

### BACKGROUND OF INVENTION

Consumers have grown to desire more interactive entertainment, such as video games, home theater systems, and 3-D movies with surround sound. Graphic novels have increased in popularity, and convenience and portability have also become important, especially with the advent of the iPod and related systems. However, to date, there has been no successful attempt to merge printed material with other forms of entertainment as it appears that consumers are reluctant to move to paperless books. Still, there is a desire to enhance the experience of reading, while not replacing the book.

### BRIEF SUMMARY

The subject invention relates to a method and apparatus for synchronization of sensory stimulation with the reading experience. In a specific embodiment, audio and/or video stimulation can be provided to a reader. In additional embodiments, the sensory stimulation can include touch, smell, and/or taste. The reading experience can include, for example, the reading of books, magazines, textbooks, telephone directories, maps, resumes, brochures, newspapers, or other forms of written or printed materials, which can include, but are not limited to, words, photographs, illustrations, drawings, cartoons, and braille. The synchronization of sensory stimulation with the reading experience can involve providing sensory stimulation in relation to a reader's location or approximate location with respect to written or printed materials. In addition to sensory stimulation, other forms of interaction can be provided, such as interactive games, or interactive tests. A specific embodiment relates to a method and apparatus for synchronizing audio and/or video to a book as it is read.

In a specific embodiment, one or more RFID chips, or tags, are embedded in certain pages of a book. The RFID tags can be active, semipassive, and/or passive, depending on the application. The turning of the pages can be detected by signals received from the RFID tags on the pages.

Another portion of the device can control the production of the sensory stimulation. In a specific embodiment, this portion of the device can control an audio and/or video system. Upon receipt of signals indicating the reader has reached certain portions, or pages, of the book, the audio and/or video system can be triggered to present certain audio and/or visual stimulation to the reader. The adjacent page or pages can have a shield or other passive or active interference device that can allow the signal to and/or from the RFID chip on a certain page to interact with the portion of the device controlling the audio and/or video system only when the book is open to that page. Upon sending and/or receiving one or more signals to and/or from the RFID chip or tag, the portion of device controlling the audio and/or video system can initiate, for example, music, scene, and/or background sounds corresponding to the page of the book that is opened.

### BRIEF DESCRIPTION OF DRAWINGS

FIG. 1 shows a schematic of an embodiment of the subject invention.

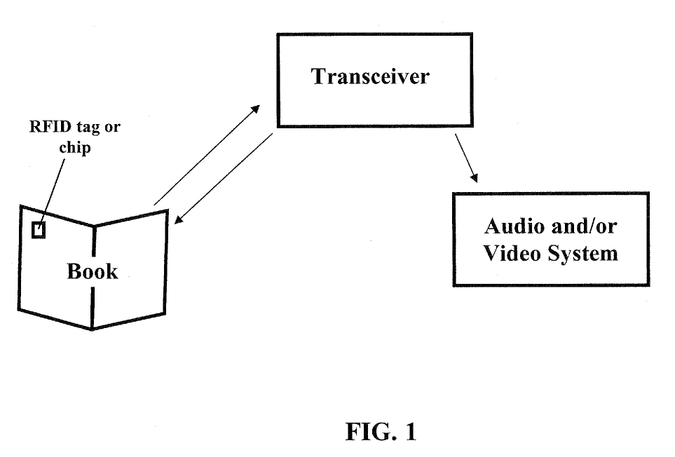

FIG. 2 shows an embodiment that incorporates one or more passive shields to “hide” a given RFID tags from an RFID tag sensor transmitter or RFID tag signal detector.

### DETAILED DISCLOSURE

The subject invention relates to a method and apparatus for synchronization of sensory stimulation with the reading experience. In a specific embodiment, audio and/or video stimulation can be provided to a reader. In additional embodiments, the sensory stimulation can include touch, smell, and/or taste. The reading experience can include, for example, books, magazines, textbooks, telephone directories, maps, resumes, brochures, newspapers, or other forms of written materials. In addition to sensory stimulation, other forms of interaction can be provided, such as interactive games, or interactive tests. A specific embodiment relates to a method and apparatus for synchronizing audio and/or video to a book as it is read.

Providing sensory stimulation to a reader of written or printed material can enhance the experience of the reader by, for example, increasing the enjoyment experienced by the reader, increasing the rate of learning or the amount of learning and/or increasing the quantity and quality of information provided to the reader. Artists and musicians can provide visual examples of their art and audio examples of their music, respectively, while the reader is reading about the artist and/or viewing photographs related to the artist. A blind person can hear the words of a book read to her, and/or any illustrations or pictures described, as she turns the pages of the book, where the book need not be in Braille. A blind person, or disabled person, can hear a Braille book read to her as she turns the pages, which can, for example, assist in the learning of Braille. A student can have a book read to him as he turns the pages, which can assist him in learning to read. Updates can be given to a reader during turning the pages in a book so that the reader can be alerted to updated news and information. The use of instructional manual or how to book, such as assembly instructions or a cook book, can be enhanced with additional sensory stimulation. A reader can have foreign language books read in certain accents or proper enunciation while turning the pages. Certain colors, sounds, meditational music, or tones can be provided while reading self-help or metaphysical books.

In a specific embodiment, one or more RFID chips, or tags, are embedded in certain pages of a book. The RFID tags can be active semipassive, and/or passive, depending on the application. Passive RFID's do not have their own power source, but rely on an external wireless transmitter to activate them. When a Passive RFID “hears” an appropriate transmitter it can transmit some of the power back as a multi-bit number. In a specific embodiment, the number could simply be a unique book id number (ISBN for example) and the page number of the page that is open. RFID's have a range, typically of a few meters. The turning of the pages can be detected by signals received from the RFID tags on the pages. With the inclusion of RFID tags, local coupling antennas, and local shields, enough information is provided to allow computer programs to sufficiently accurately determine the specific RFID tags and their relative locations, or separations so as to allow a determination of position in the written or printed materials the reader is at.

Embodiments of the invention can also be used to gather information regarding the reader's interaction with the written or printed material, such as how fast the reader progresses through the written or printed material, with or without providing the reader sensory stimulation. Such information can be used to assist teachers assess the reader's (students) study habits, reading speed, or assist advertisers determine how long readers look at certain pages provided to them.

For an additional embodiment, which can be useful for blind readers, the signal elements in the written materials can include the sensory stimulation data. As an example, the signal elements in the written materials can include precisely the information printed on the page such that the sensory stimulation device does not have to have previously stored data or need to get the data from another source, such as the internet.

In a specific embodiment, character recognition can be used to gather information on to the position the reader is at in the written or printed material. Cameras can be positioned to view the printed material. Such cameras can be mounted such that a view similar to the view that a person has of the written material can search the pages for, for example, key words or page numbers to activate the devices providing sensory input. In a specific example, cameras can be connected to one or both sides of a pair of glasses that then are connected to a computer system (either wired or non-wired) that runs software that enables identification of key words, chapter headings, or page numbers to produce electronic control of audio/visual displays. Web cams can also be used. Webcams can allow the use of internet programs to detect the key words or page numbers and/or provide the sensory stimulation.

Simple tuned circuits can be embedded in or attached to pages in such a way that resultant resonant frequencies indicate the relative proximities of a multitude of pages, as coupled resonant circuits can produce splitting related to the separation of the circuits, enabling inversion to the approximate page a book or magazine has open. A frequency sweeping detector can be placed in close proximity to produce the frequency spectrum. Automated software can analyze the spectrum and decide upon the most likely open page and then through connection to a computer system produce the results described above. A device can include accelerometers and/or localized positioning systems such as GPS, and can be attached to a readers hand to count the pages turned by detecting the “typical” motion that results from turning a page with ones hand. If it is assumed that every page will be viewed, and furthermore that the reader could select between two options of starting at the first page, or starting where they last left off, then, in principle, the page open would always be known.

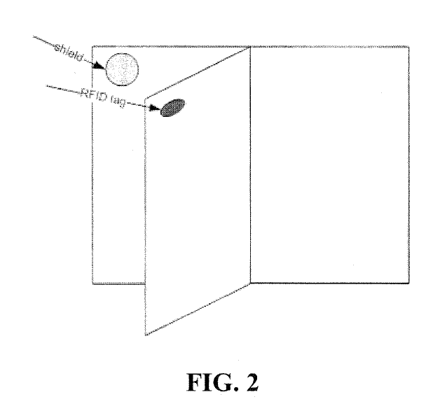

Another portion of the device can control the production of the sensory stimulation. In a specific embodiment, this portion of the device can control an audio and/or video system. In other embodiments, this portion can control taste, touch, and/or smell stimulation provided in the environment of the reader. Although the specific embodiments of audio and/or visual are used to describe an exemplary embodiment, the teachings apply to the other sensory stimulation as well. Upon receipt of signals indicating the reader has reached certain portions, or pages, of the book, the audio and/or video system can be triggered to present certain audio and/or visual stimulation to the reader. The adjacent page or pages can have a shield or other passive or active interference device that can allow the signal to and/or from the RFID chip on a certain page to interact with the portion of the device controlling the audio and/or video system only when the book is open to that page or section. Referring to a specific embodiment shown in FIG. 2, passive shields can be utilized on adjacent pages to “hide” a given RFID tag from the RFID sensor transmitter or from the RFID signal detector. The effectiveness of shields, such as a solid piece of conductor, e.g., aluminum foil, depends upon how close the shield is to the RFID, in comparison to the size of the RFID and how large the shield is in comparison to the size of the RFID. Detecting the page can be accomplished by the RFID being seen for a relatively short amount of time, for example, as the page is turned, the RFID is active and then stops again as it comes near a shield on the previous page. In this method each RFID is sandwiched between two shields. Two or more RFID tags can be “unshielded” when a particular page is open. This uses redundancy to improve the accuracy of detection. An embodiment can also require that all tags are seen or a large majority are seen by the RFID tag reader.

Upon sending and/or receiving one or more signals to and/or from the RFID chip or tag, the portion of device controlling the audio and/or video system can initiate, for example, music, scene, and/or background sounds corresponding to the page of the book that is opened. In a specific embodiment, smart house controls, such as lighting, sound systems, video displays, and/or fountains, can be activated, deactivated, controlled, and/or dimmed, by the sensory stimulation device based on the position in the written or printed material being viewed.

One example of the portion of the device for controlling the audio and/or video is an RF transceiver that upon detection of a particular RFID chip or tag, sends an activation signal to another device, such as an mp3 player or an iPod™. The other device can then play, for example, a particular track of sound or music, concurrently with the reader's progress in the book. Other examples of the enhancement of the reading experience can include using the sound track of a movie played at the appropriate times during the related actions in the book and playing background sound on a home audio system, such as crickets for an evening natural setting. Specific embodiments can incorporate features, such as smart processing, based upon this. Smart processing can use the reader's pace to more closely associate the audio and/or visual experience with the particular action or scene in the book. Other “fill-in” sounds can be used to create an appropriate mood, and to place a key piece of music more exactly at the time the reader is encountering the passages in the book. Embodiments also allow interfacing with computers, the internet, or other sources of information, which can be updated as needed. The book can provide a trigger to go to a certain website.

In specific embodiments, video can be incorporated into the experience. As an example, synchronized background scenery can be provided on a large screen near the reader. As another example, if a scene in a book takes place at a waterfront location, a video of a lake shore can be shown on a screen. “Hidden tracks” may be put in by the author in which the author tells the reader to pause reading while information not completely necessary to the story, such as interesting background data, is related. Control of internet audio and/or video data may also be accomplished. Another example of the use of embodiments of the subject system is for educational books, in which lectures, diagrams, and/or photographs, can be presented as the reader arrives at the appropriate location in the book. With internet control this can allow a single book to maintain current information status, with new editions perhaps being only changes in the material not printed in the book, but, instead, changes to the audio and/or video portions.

In additional embodiments, the control system for synchronization of audio and/or video with the progress in the book can be performed using light sensitive devices, so that when the book is opened to a particular page, or pair of pages, the light activates a device that sends a signal to a nearby device that controls the audio and/or video signals. In an embodiment, the light sensing device can be placed near the spine of the book, so that only the correct device activates the controls. Additionally, for either optical or RF systems, the secondary device that receives the signal from the book can have methods for choosing the correct page if multiple signals are detected. For example, the device may assume that the book is being read from front to back, and only the next signal may be permitted. Alternatively there can be a signal strength comparison, such that, for example, the optical device sensing the most light, is assumed to indicate the correct page. A combination of these devices and/or methods may be appropriate as well. For example, the optical sensor or detector, can be used in series with the RFID circuit, such that an identification of the position is not made unless both positive signals are received. The optical detector could receive a signal based on whether there is a threshold amount of ambient light, such as enough light to read, or enough light reflecting from a certain page to indicate the written or printed material is opened to that page. This should be very effective when the RFID and sensor were relatively near the spine of the book, instead of near the outside margins of the book. The optical sensor can be on the outside surface of the page.

Additional embodiments can use redundancy to improve the accuracy of the signal. In addition to embodiments using RFID signals and optical signals for redundancy, often redundancy can use two RFID tags, for example, on opposite pages, that the signals of both used to be received for a positive identification of a certain position of the written or printed materials to be made. Specific embodiments can include a feedback interface for the reader to provide feedback to sensory stimulation. As an example, an audio signal can be presented to the reader that states to press the “yes” button if at page 171 and to press “no” if not at page 171. As another example, the audio stimulation could ask the reader to enter the page number the reader is at and the interface could allow the reader to enter such page number. In a specific embodiment, the reader can provide feedback to slow down an audio track, such as when the device is being used to teach a student to read.

In a further embodiment, a device that allows the audio and/or visual book system to perform eye tracking can be utilized. This permits nearly precise synchronization with the reader. By observing the eye movement, the location in the text, line by line can be tracked at all times. This allows for highly accurate timing of the audio and/or visual experience, such as, crescendo music swelling at a time of great excitement or a gun shot when the reader reads “a shot rang out”.

In many cases, it is presumed that a book author will create or participate in the creation of the concomitant audio and/or visual elements. In principle, however, the audio and/or visual experience open to the reader may be independent of the books, such that several different artists may produce music imagery or video for a given book. Then the book “experiencer” may choose an artist they believe will provide the experience most interesting to them. It may also be desirable to permit advertising in the form of the selection of the audio and/or visual experience. As an example, suppose the book mentions that a car goes by, then an auto manufacturer may pay for the car in the audio and/or visual portion to be one of their automobiles. These elements can change over time, dependent upon the sponsor, without significantly affecting the viewer's experience of the book, while providing an advertising service. Direct advertising can also be utilized.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application.

1. A system for providing sensory stimulation to a reader of a written or printed material, comprising:
a position detector, wherein the position detector detects the position of a written or printed material a reader is at; and
a sensory stimulation device, wherein the sensory stimulation device provides sensory stimulation to the reader based on the position of the written or printed material the reader is at.

2. The system according to claim 1, wherein the position detector comprises at least one signaling element positioned proximate the written or printed material, wherein the at least one signaling element provides a corresponding at least one signal indicating the position of the written or printed material the reader is at.

3. The system according to claim 2, wherein the at least one signal is a wireless signal.

4. The system according to claim 1, wherein the position of the written or printed material is a page of the written or printed material the written or printed material is open to.

5. The system according to claim 1, wherein the written or printed material is a book.

6. The system according to claim 1, wherein the written or printed material is selected from the group consisting of, a magazine, a textbook, a telephone directory, a map, a brochure, a newspaper, and a resume.

7. The system according to claim 1, wherein the sensory stimulation is audio stimulation.

8. The system according to claim 1, wherein the sensory stimulation comprises audio and/or visual stimulation.

9. The system according to claim 1, wherein the sensory stimulation comprises one or more of the following: taste stimulation, smell stimulation, and touch stimulation.

10. The system according to claim 2, wherein the at least one signaling element comprises:
at least one RFID tag attached to the written or printed material.

11. The system according to claim 10, wherein the position detector further comprises an RFID tag signal sensor detects the at least one signal from the at least one RFID tag.

12. The system according claim 11, wherein the position detector further comprises and RF excitation source, wherein, upon excitation by the RF excitation source, one or more of the at least one RFID tag provides a corresponding one or more signals indicating the position of the written or printed material the reader is at.

13. The system according to claim 10, wherein one or more of the at least one RFID tag is active.

14. The system according to claim 10, further comprising:
at least one active or passive shield for preventing detection of a signal from one or more of the at least one RFID tag until the reader is at a corresponding position of the written or printed material.

15. The system according to claim 1, wherein the sensory stimulation comprises an interactive sensory stimulation.

16. The system according to claim 1, wherein the sensory stimulation is related to the situation described in the written or printed material.

17. The system according to claim 1, wherein the position detector comprises:
at least one light detector, wherein the at least one light detector is positioned to receive light from the written or printed material.

18. The system according to claim 17, wherein the at least one light detector comprises one or more cameras.

19. The system according to claim 18, wherein the position detector further comprises character recognition, wherein recognized characters from the written or printed material correspond to the position of the written or printed material the reader is at.

20. The system according to claim 10, wherein one or more of the at least one RFID tag is embedded in one or more pages of the written or printed material.

21. The system according to claim 1, further comprising:
at least one signaling element, wherein the at least one signaling element provides at least one signal providing information with respect to sensory stimulation corresponding to a position in a written or printed material a reader is at; and
a detector, wherein the detector detects the at least one signal, wherein the sensory stimulation device provides a sensory stimulation to the reader based on the information with respect to sensory stimulation provided in the detected at least one signal.

22. The system according to claim 1, further comprising:
a reader interface, wherein the reader interface allows the reader to provide feedback to the sensory stimulation.

23. The system according to claim 1, wherein the position detector comprises at least one tuned circuit attached to the written or printed material, wherein resonant frequencies of the at least one tuned circuit indicates the position in the written or printed material.

24. The system according to claim 21, wherein the at least one signaling element comprises one or more of the following: an accelerometer, a localized positioning device, and a GPS device.

25. The system according to claim 1, wherein the sensory stimulation device controls one or more of the following: smart house controls, lighting, and fountains.

26. The system according to claim 1, wherein the sensory stimulation is received via a communication channel that allows receipt of information from a remote location.

27. The system according to claim 26, wherein the communication channel utilizes the internet.

28. The system according to claim 1, further comprising:
a light sensing device, wherein the light sensing device is positioned proximate the written or printed material, wherein the light sensing device determines whether there is enough light to read.

29. The system according to claim 1, wherein the position detector comprises an eye tracking detector, wherein the eye tracking detector detects movement and/or position of one or both eyes of the reader.

30. A system for providing sensory stimulation to a reader of a written or printed material, comprising:
at least one signaling element, wherein the at least one signaling element provides at least one signal providing information with respect to sensory stimulation corresponding to a position in a written or printed material a reader is at;
a detector, wherein the detector detects the at least one signal; and
a sensory stimulation device, wherein the sensory stimulation device provides a sensory stimulation to the reader based on the information with respect to sensory stimulation provided in the detected at least one signal.

31. The system according to claim 30, where the at least one signaling element comprises at least one RFID tag.

32. A method for enhancing a reading experience, comprising:
providing a reader written material;
determining a portion of the written material the reader is at;
providing sensory stimulation to the reader based on the portion of the written material the reader is at.

33. A method for providing sensory stimulation to a reader of a written or printed material, comprising:
detecting the position of a written or printed material a reader is at; and
providing sensory stimulation to the reader based on the position of the written or printed material the reader is at.

34. The method according to claim 33, wherein detecting the position of a written or printed material comprises positioning at least one signaling element proximate the written or printed material, wherein the at least one signaling element provides a corresponding at least one signal indicating the position of the written or printed material the reader is at; and detecting the at least one signal indicating the position of the written or printed material the reader is at.

35. The method according to claim 34, wherein the at least one signal is a wireless signal.

36. The method according to claim 33, wherein the position of the written or printed material is a page of the written or printed material the written or printed material is open to.

37. The method according to claim 33, wherein the written or printed material is a book.

38. The method according to claim 33, wherein the written or printed material is selected from the group consisting of, a magazine, a textbook, a telephone directory, a map, a brochure, a newspaper, and a resume.

39. The method according to claim 33, wherein the sensory stimulation is audio stimulation.

40. The method according to claim 33, wherein the sensory stimulation comprises audio and/or visual stimulation.

41. The method according to claim 33, wherein the sensory stimulation comprises one or more of the following: taste stimulation, smell stimulation, and touch stimulation.

42. The method according to claim 34, wherein the at least one signaling element comprises:
at least one RFID tag attached to the written or printed material.

43. The method according to claim 42, wherein the detecting the at least one signal comprises detecting the at least one signal from the at least one RFID tag.

44. The method according claim 43, further comprising exciting one or more of the at least one RFID tag, wherein exciting the one or more of the at least one RFID tag provides a corresponding one or more signals indicating the position of the written or printed material the reader is at.

45. The method according to claim 42, wherein one or more of the at least one RFID tag is active.

46. The method according to claim 42, further comprising:
preventing detection of a signal from one or more of the at least one RFID tag by shielding the one or more of the at least one RFID tag until the reader is at a corresponding position of the written or printed material.

47. The method according to claim 33, wherein the sensory stimulation comprises an interactive sensory stimulation.

48. The method according to claim 33, wherein the sensory stimulation is related to the situation described in the written or printed material.

49. The method according to claim 33, wherein detecting the position in a written or printed material a reader is at comprises:
positioning at least one light detector to receive light from the written or printed material; and
detecting the position via at least one light detector.

50. The method according to claim 49, wherein the at least one light detector comprises one or more cameras.

51. The method according to claim 50, wherein detecting the position in a written or printed material a reader is at comprises performing character recognition on the light, wherein recognized characters from the written or printed material correspond to the position of the written or printed material the reader is at.

52. The method according to claim 42, wherein one or more of the at least one RFID tag is embedded in one or more pages of the written or printed material.

53. The method according to claim 33, wherein detecting the position in a written or printed material a reader is at comprises:
positioning at least one signaling element, wherein the at least one signaling element provides at least one signal providing information with respect to sensory stimulation corresponding to a position in a written or printed material a reader is at; and
detecting the at least one signal, wherein providing sensory stimulation to the reader comprises providing sensory stimulation to the reader based on the information with respect to sensory stimulation provided in the detected at least one signal.

54. The method according to claim 33, further comprising:
providing a reader interface, wherein the reader interface allows the reader to provide feedback to the sensory stimulation.

55. The method according to claim 33, wherein detecting the position of the written or printed material the reader is at comprises:
attaching at least one tuned circuit detecting to the written or printed material; and
resonant frequencies of the at least one tuned circuit, wherein the detected resonant frequencies indicate the position in the written or printed material.

56. The method according to claim 53, wherein the at least one signaling element comprises one or more of the following: an accelerometer, a localized positioning device, and a GPS device.

57. The method according to claim 33, wherein providing sensory stimulation comprises controlling one or more of the following: smart house controls, lighting, and fountains.

58. The method according to claim 33, wherein the sensory stimulation is received via a communication channel that allows receipt of information from a remote location.

59. The method according to claim 58, wherein the communication channel utilizes the internet.

60. The method according to claim 33, further comprising:
positioning a light sensing device proximate the written or printed material,
determining whether there is enough light to read via the light sensing device.

61. The method according to claim 33, wherein detecting the position of the written or printed material the reader is at comprises a tracking eye movement and/or position of one or both eyes of the reader.

62. A method for providing sensory stimulation to a reader of a written or printed material, comprising:
providing at least one signaling element, wherein the at least one signaling element provides at least one signal providing information with respect to sensory stimulation corresponding to a position in a written or printed material a reader is at;
detecting the at least one signal; and
providing sensory stimulation to the reader based on the information with respect to sensory stimulation provided in the detected at least one signal.

63. The method according to claim 62, where the at least one signaling element comprises at least one RFID tag.
